(19) United States
(12) Patent Application Publication
(10) Pub. No.: US 2026/0174743 A1
(43) Pub. Date: Jun. 25, 2026

(54) METHODS FOR STABILIZING LIQUID BACTERIAL ENDOSPORE COMPOSITIONS

(71) Applicants: BIOWISH TECHNOLOGIES, INC., Cincinnati, OH (US); SABIC AGRI-NUTRIENTS COMPANY SJSC, Qurtubah (SA)

(72) Inventors: William Howard DIEDERICH, Cincinnati, OH (US); John P. GORSUCH, Cincinnati, OH (US); Munif Hamad Nasser AL-MUNIF, Qurtubah (SA); Radwan M. ABDALLAH, Qurtubah (SA); James Edward HAIGH, JR., Qurtubah (SA)

(21) Appl. No.: 19/128,793

(22) PCT Filed: Nov. 9, 2023
(86) PCT No.: PCT/US2023/079253

§ 371 Date: May 9, 2025

Related U.S. Application Data
(60) Provisional application No. 63/424,643, filed on Nov. 11, 2022.

(51) Int. Cl. A61K 31/4439; A61K 31/513; A61K 31/5415; A61K 45/06

(52) U.S. Cl. CPC A61K 31/4439; A61K 31/513; A61K 31/5415; A61K 45/06; A01N 2300/00; A61K 2300/00

(57) ABSTRACT

The present invention relates to methods for stabilizing liquid bacterial endospores compositions. The present invention also relates to multi-functional osmotic preservation strategies for liquid Bacillus endospore suspensions.

### CROSS REFERENCE TO RELATED APPLICATIONS

This application claims priority to, and benefit of, U.S. Provisional Patent Application No. 63/424,643 filed on Nov. 11, 2022, the contents of which are incorporated by reference in their entirety herein.

### FIELD OF THE INVENTION

The present invention relates to methods for stabilizing liquid bacterial endospore compositions. The present invention also relates to multi-functional osmotic preservation strategies for liquid Bacillus endospore suspensions.

### BACKGROUND OF THE INVENTION

Endospores are metabolically dormant, durable, non-reproductive cells formed by bacteria of the phylum Bacillota (basonym Firmicutes) in response to nutrient-limited or otherwise unfavorable environmental conditions. Endospore-forming genera include Bacillus, Brevibacillus, Lysinibacillus, and Paenibacillus among many others. Endospores are resistant to a variety of physical and chemical stressors that would kill vegetative bacterial cells, including extreme temperatures, chemical disinfectants, desiccation, and ultraviolet radiation. Because of this resilience, endospores (especially formed by members of the genus Bacillus) are popular inclusions in microbial biostimulant and biofertilizer preparations. Because liquid endospore suspensions are more easily integrated into unit operations in fertilizer coating facilities and growers' management practices, they may be considered commercially favorable to lyophilized and spray-dried powders. However, concentrated liquid endospore suspensions are known to face several challenges which impact product stability. These suspensions often contain high concentrations of biomolecules left over from the fermentation process (J. P. Gorsuch, Z. Jones, Heliyon 6 (2020) e03419) which can support the growth of heterotrophic environmental spoilage organisms in the absence of an aggressive antimicrobial preservation package (Setlow, P., J. Appl. Microbiol. 126, 348-358, 2018). Although endospores are more resistant to chemical stressors than vegetative bacterial cells, many may be labile to concentrated preservative levels given sufficient contact time (Setlow, P., J. Bacteriol. 196, 1297-1305, 2014). Therefore, a preservative system which prohibits the proliferation of spoilage organisms without compromising the viability of the product's formulated endospores is essential. Additionally, Bacillus endospores may be vulnerable to non-nutrient germination, in which a biomolecule such as peptidoglycan or dipicolinic acid triggers a germination response which robs the endospore of its trademark metabolic dormancy and associated resistance to environmental and chemical stressors (Setlow, P., J. Bacteriol. 196, 1297-1305, 2014). Because such biomolecules may be present in concentrated industrial endospore suspensions (J. P. Gorsuch, Z. Jones, Heliyon 6 (2020) e03419), a preservative system which mitigates the non-nutrient germination response of labile endospores is also essential to product stability. Such a system may function either chemically (through the direct inactivation of non-nutrient germinant compounds) or biologically (through management of the endospore's non-nutrient germination response).

Products such as BiOWiSH® Crop Liquid™, a 1:1:1:1 blend of four Bacillus species: Bacillus amyloliquefaciens (BA). B. subtilis (BS), B. pumilus (BP), and B. licheniformis (BL), are subject to spoilage by environmental microorganisms and loss of endospore viability over time under stressed storage conditions. While advanced chemical preservative packages adequately protect the product from spoilage by environmental contaminants, they also have a negative effect on endospore stability. Therefore, there exists a need in the art for a preservative system which can mitigate the threat of spoilage posed by hardy environmental contaminants, while also improving stability outcomes for labile Bacillota endospores.

### SUMMARY OF THE INVENTION

Described herein are compositions and methods for extending storage time and reducing spoilage of a liquid endospore suspension. The present disclosure provides an optimal preservative system for a liquid endospore suspension, which produces a physical and chemical environment within the suspension that is antagonistic to spoilage organisms while simultaneously supportive of endospore dormancy and stability. This in turn allows the product to be shipped and stored for extended periods of time, without special handling requirements, to inhibit or delay the growth of environmental spoilage organisms such as bacteria, yeast, fungi, viruses, and/or pests.

Accordingly, in one aspect the present disclosure provides a composition comprising a liquid endospore suspension and at least one osmotic preservative.

In some embodiments, the liquid endospore suspension is an aqueous suspension.

In some embodiments, the liquid endospore suspension comprises at least one endospore forming Bacillota.

In some embodiments, the at least one endospore forming Bacillota is selected from the group consisting of Bacillus, Brevibacillus, Paenibacillus, and Lysinibacillus genera.

In some embodiments, the Bacillus species is selected from the group consisting of B. amyloliquefaciens (BA), B. subtilis (BS), B. pumilus (BP), B. licheniformis (BL). B. thuringiensis, B. velezensis, B. endophyticus, and Priestia megaterium (basonym Bacillus megaterium).

In some embodiments, the at least one osmotic preservative is an osmolarity modifier. Further, in some embodiments, the osmolarity modifier reduces water energy and/or induces preservation of the endospores.

In some embodiments, the at least one osmotic preservative is a chloride salt. Further, in some embodiments, the at least one osmotic preservative is selected from the group consisting of sodium chloride, calcium chloride, potassium chloride, magnesium chloride and any other chloride salt.

In some embodiments, the composition comprises at least about 10.0 wt % of the at least one osmotic preservative.

In some embodiments, the liquid endospore suspension is stable at 40° C. for at least 180 days.

Another aspect of the present disclosure provides a method of inhibiting the proliferation of at least one environmental spoilage organism in a liquid endospore suspension, wherein the method comprises use of at least one osmotic preservative.

In some embodiments, the at least one environmental spoilage organism is selected from a bacterial, a yeast, or a fungus contaminant. Further, in some embodiments, the bacterial contaminant is selected from the group consisting of Pseudomonas, Citrobacter, Klebsiella, Shigella, Enterobacter, and Serratia species.

In some embodiments, the at least one endospore forming Bacillota is selected from the group consisting of Bacillus, Brevibacillus, Paenibacillus, and Lysinibacillus genera. In some embodiments, at least one endospore forming Bacillota comprises a species from a genus selected from the group consisting of Bacillus, Brevibacillus, Paenibacillus, and Lysinibacillus.

In some embodiments, the Bacillus species is selected from the group consisting of B. amyloliquefaciens (BA), B. subtilis (BS), B. pumilis (BP), B. licheniformis (BL), B. thuringiensis, B. velezensis, B. endophyticus, and Priestia megaterium (basonym Bacillus megaterium).

In some embodiments, the at least one osmotic preservative is an osmolarity modifier. Further, in some embodiments, the osmolarity modifier reduces water energy and/or induces preservation of the endospores.

In some embodiments, the at least one osmotic preservative is selected from the group consisting of sodium chloride, calcium chloride, potassium chloride, magnesium chloride and any other chloride salt.

In some embodiments, the liquid endospore suspension is stable at 40° C. for at least 180 days.

### BRIEF DESCRIPTION OF THE DRAWINGS

FIG. 1 shows spoilage organisms recovered from samples of a commercially available endospore suspension protected with a standard chemical preservative and exposed to standard unit operations at a customer's facility, necessitating the inclusion of an advanced antimicrobial inhibitor system in future product iterations.

FIG. 2 shows stability of a model endospore blend under stressed conditions in the presence of a standard chemical preservative. BA: B. amyloliquefaciens; BS: B. subtilis; BP: B. pumilus; BL: B. licheniformis.

FIG. 3 shows mitigation of endospore suspension spoilage by environmental microorganisms through the use of advanced chemical preservation packages. Success of chemical preservation strategies was assessed using a customized Preservative Efficacy Test.

FIG. 4 shows a stability of a model endospore blend under stressed conditions in the presence of an advanced chemical preservative. Sub-populations of individual species within the blend were assessed using a customized spread-plate assay with morphological scoring allowing separate reporting of each population from a single tested blend.

FIG. 5 shows morphological scoring of an APC plate (a), allowing populations of individual species BA (b), BS (c), BP (d), and BL (e) to be evaluated within a mixed-species suspension.

FIG. 6 shows stability of BA endospores in various osmotic preservative systems.

FIG. 7 shows stability of BS endospores in various osmotic preservative systems.

FIG. 8 shows stability of BP endospores in various osmotic preservative systems.

FIG. 9 shows stability of BL endospores in various osmotic preservative systems.

### DETAILED DESCRIPTION OF THE INVENTION

The present disclosure relates to an optimal preservative system for a liquid endospore suspension (i.e. aqueous endospore suspension), which produces a physical and chemical environment within the suspension that is antagonistic to spoilage organisms while simultaneously supportive of endospore dormancy and stability. Advantages of a preservative system for a liquid endospore suspension include, but is not limited to, the mitigation of the threat of spoilage posed by hardy environmental contaminants while also improving stability outcomes for labile Bacillus endospores. Compositions of the present disclosure can extend storage time, reduce spoilage of a liquid endospore suspension, and support endospore dormancy and stability.

One aspect of the present disclosure relates to a composition comprising a liquid endospore suspension and at least one osmotic preservative. A related aspect of the present disclosure relates to a composition comprising an aqueous endospore suspension and at least one osmotic preservative. Notably, the preservative system comprising at least one osmotic preservative produces a physical and chemical environment within the liquid endospore suspension that is antagonistic to spoilage organisms while simultaneously supportive of endospore dormancy and stability.

In some embodiments, the liquid endospore suspension comprises at least one endospore forming Bacillota. In some embodiments, the at least one endospore forming Bacillota is from genus Bacillus, Brevibacillus, Paenibacillus, Lysinibacillus, or another Bacillota genus. In some embodiments, the at least one endospore forming Bacillota is a species within the genera Bacillus, Brevibacillus, Paenibacillus, or Lysinibacillus. In some embodiments, the Bacillus species is B. amyloliquefaciens (BA), B. subtilis (BS), B. pumilus (BP), B. licheniformis (BL), B. thuringiensis. B. velezensis, B. endophyticus, Priestia megaterium (basonym Bacillus megaterium), or another Bacillus species. In some embodiments, the Bacillus species is B. amyloliquefaciens (BA), B. subtilis (BS), B. pumilus (BP), or B. licheniformis (BL).

In some embodiments, the liquid endospore suspension comprises one or more endospore forming Bacillota. In some embodiments, the one or more endospore forming Bacillota is a species from genus Bacillus, Brevibacillus, Paenibacillus, Lysinibacillus, another Bacillota genus, or any combination thereof. In some embodiments, the one or more endospore forming Bacillota is a species from genus Bacillus, Brevibacillus, Paenibacillus, or Lysinibacillus. In some embodiments, the Bacillus species is B. amyloliquefaciens (BA), B. subtilis (BS), B. pumilus (BP), B. licheniformis (BL), B. thuringiensis. B. velezensis, B. endophyticus, Priestia megaterium (basonym Bacillus megaterium), another Bacillus species, or any combination thereof. In some embodiments, the Bacillus species is B. amyloliquefaciens (BA), B. subtilis (BS), B. pumilus (BP), or B. licheniformis (BL) or a combination thereof. In some embodiments, the B. amyloliquefaciens (BA), B. subtilis (BS), B. pumilus (BP), and B. licheniformis (BL) spores are present in equal concentrations in the liquid endospore suspension as determined by colony forming units (CFU). In some embodiments, the B. amyloliquefaciens (BA), B. subtilis (BS), B. pumilus (BP), and B. licheniformis (BL) spores are not present in equal concentrations in the liquid endospore suspension as determined by CFU.

Bacillota species such as Bacillus, Brevibacillus, Paenibacillus, Lysinibacillus genus species are known to persons of ordinary skill in the art. Such species are available from generally recognized depositories. Exemplary deposit information for bacterial species that can be used in the liquid endospore suspensions of the disclosure is provided in Table A below.

TABLE A
Deposit Numbers for Exemplary bacteria species

| | Deposit | |
|---|---|---|
| Species | Institution | Deposit No(s). |
| B. amyloliquefaciens | ATCC* | 23350, BAA-390, 23842, |
| | | 23844 |
| B. subtilis | ATCC | 6051, 14416, 14410, 14415, |
| | | 10783 |
| B. pumilus | ATCC | 7061, 700814, 4520, BAA- |
| | | 1434 |
| B. licheniformis | ATCC | 14580, 12713, 9945 |
| B. thuringiensis | ATCC | 10792, 33679, 29730, |
| | | 35646, 35866 |
| B. velezensis | CGMCC** | cgmcc 1.923 |
| B. endophyticus | JCM*** | 12211 |
| Priestia megaterium | ATCC | 14581, 9885, 19213, 12872, |
| | | 19380, 39383 |

*American Type Culture Collection (atcc.org)
**The Chinese General Microbiological Culture Collection Center (cgmcc.net)
***JCM On-line Catalog of Strains (RIKEN BRC Microbe Division, jem.brc.riken.jp)

In some embodiments, liquid endospore suspension has a microbial concentration from about 1×10⁶ to about 1×10¹² colony forming units per mL (CFU/mL), e.g., 1×10⁶ to about 1×10¹¹ CFU/mL, about 1×10⁸ to 1×10¹² CFU/mL, or about 1×10⁹ to 1×10¹² CFU/mL. In some embodiments, the liquid endospore has a microbial concentration comprising about 1×10⁷ to about 1×10¹¹ CFU/mL. In some embodiments, the liquid endospore has a microbial concentration comprising about 1×10⁸ to about 1×10¹⁰ CFU/mL. In some embodiments, the liquid endospore has a microbial concentration comprising about 5×10⁸ to about 1×10¹⁰ CFU/mL. In some embodiments, the liquid endospore has a microbial concentration comprising about 5×10⁸ to about 5×10⁹ CFU/mL.

In some embodiments, the at least one osmotic preservative is an osmolarity modifier. In some embodiments, the at least one osmotic preservative is a combination of osmolarity modifiers. In some embodiments, the osmolarity modifier increases osmolarity within the liquid endospore suspension. In some embodiments, the osmolarity modifier decreases osmolarity within the liquid endospore suspension. In some embodiments, the osmolarity modifier reduces water energy and/or induces preservation of the endospores within the liquid endospore suspension.

In some embodiments, the preservative system can include at least one osmotic preservative, at least two osmotic preservatives, at least three osmotic preservatives, or four or more osmotic preservatives. In some embodiments, the preservative system can include at least one osmolarity modifier, at least two osmolarity modifiers, at least three osmolarity modifiers, or four or more osmolarity modifiers. Alternatively, in some embodiments, the preservative system can be a chemical and/or physical preservative system.

In some embodiments, the at least one osmotic preservative comprises a salt or a combination of salts. In some embodiments, the at least one osmotic preservative comprises one or more chloride salts. In some embodiments, the at least one osmotic preservative comprises sodium chloride, calcium chloride, potassium chloride, magnesium chloride, or any combination thereof. In some embodiments, the osmotic preservative increases osmolarity within the liquid endospore suspension. In some embodiments, the osmotic preservative decreases osmolarity within the liquid endospore suspension. In some embodiments, the osmotic preservative reduces water energy and/or induces preservation of the endospores within the liquid endospore suspension.

In some embodiments, the preservative system can include at least one osmotic preservative, at least two osmotic preservatives, at least three osmotic preservatives, or four or more osmotic preservatives. In some embodiments, the preservative system can include at least one salt, at least two salts, at least three salts, or four or more salts. Alternatively, in some embodiments, the preservative system can be a chemical and/or physical preservative system.

In some embodiments, the composition comprising a liquid endospore suspension and at least one osmotic preservative can remain stable at about 20° C. to about 40° C. for at least 3 months, at least 4 months, at least 5 months, at least 6 months, at least 7 months, at least 8 months, at least 9 months, at least 10 months, at least 11 months, at least a year, at least 1.5 years, or at least 2 years. In some embodiments, the stable aqueous microbial composition can remain stable at about 20° C. to about 40° C. for about 1 month to about 2 years, about 2 months to about 1.5 years, about 3 months to about 1 year, about 3 months to about 8 months, about 6 months to about 3 years, e.g., about 6 months to about 2 years, about 6 months to about 1.5 years, about 6 months to about 1 year, about 1 year to about 3 years, about 1 year to 2 years, or about 1 year to 1.5 years. In some embodiments, the composition comprising a liquid endospore suspension and at least one osmotic preservative can remain stable at about 20° C. to about 40° C. for about 3 months, about 6 months, about 9 months, about a year, about 1.5 years, or about 2 years. For example, the composition comprising a liquid endospore suspension and at least one osmotic preservative can remain stable at about 40° C. for about 6 months (i.e. 180 days).

In some embodiments, the composition comprising a liquid endospore suspension and at least one osmotic preservative can remain stable at room temperature for at least 3 months, at least 4 months, at least 5 months, at least 6 months, at least 7 months, at least 8 months, at least 9 months, at least 10 months, at least 11 months, at least a year, at least 1.5 years, or at least 2 years. In some embodiments, the composition comprising a liquid endospore suspension and at least one osmotic preservative can remain stable at room temperature for about 1 month to about 2 years, about 2 months to about 1.5 years, about 3 months to about 1 year, about 3 months to about 8 months, about 6 months to about 3 years, e.g., about 6 months to about 2 years, about 6 months to about 1.5 years, about 6 months to about 1 year, about 1 year to about 3 years, about 1 year to 2 years, or about 1 year to 1.5 years. For example, the composition comprising a liquid endospore suspension and at least one osmotic preservative can remain stable at room temperature for about 3 months, about 6 months, about 9 months, about a year, about 1.5 years, about 2 years, about 2.5 years, or about 3 years.

In some embodiments, the composition comprising a liquid endospore suspension and at least one osmotic preservative can remain biologically stable at about 20° C. to about 40° C. for at least 3 months, at least 4 months, at least 5 months, at least 6 months, at least 7 months, at least 8 months, at least 9 months, at least 10 months, at least 11 months, at least a year, at least 1.5 years, or at least 2 years. In some embodiments, the stable aqueous microbial composition can remain biologically stable at about 20° C. to about 40° C. for about 6 months to about 3 years, e.g., about 1 month to about 2 years, about 2 months to about 1.5 years, about 3 months to about 1 year, about 3 months to about 8 months, about 6 months to about 2 years, about 6 months to about 1.5 years, about 6 months to about 1 year, about 1 year to about 3 years, about 1 year to 2 years, or about 1 year to 1.5 years. In some embodiments, the composition comprising a liquid endospore suspension and at least one osmotic preservative can remain biologically stable at about 20° C. to about 40° C. for about 3 months, about 6 months, about 9 months, about a year, about 1.5 years, or about 2 years. For example, the composition comprising a liquid endospore suspension and at least one osmotic preservative can remain biologically stable at about 40° C. for about 6 months (i.e. 180 days).

In some embodiments, the composition comprising a liquid endospore suspension and at least one osmotic preservative can remain biologically stable at room temperature for at least 3 months, at least 6 months, at least 7 months, at least 8 months, at least 9 months, at least 10 months, at least 11 months, at least a year, at least 1.5 years, or at least 2 years. In some embodiments, the composition comprising a liquid endospore suspension and at least one osmotic preservative can remain biologically stable at room temperature for about 6 months to about 3 years, e.g., about 6 months to about 2 years, about 6 months to about 1.5 years, about 6 months to about 1 year, about 1 year to about 3 years, about 1 year to 2 years, or about 1 year to 1.5 years. For example, the composition comprising a liquid endospore suspension and at least one osmotic preservative can remain biologically stable at room temperature for about 3 months, about 6 months, about 9 months, about a year, about 1.5 years, about 2 years, about 2.5 years, or about 3 years.

In some embodiments, the composition comprising a liquid endospore suspension and at least one osmotic preservative can remain biologically stable for the aforementioned periods of time under a variety of humidity storage conditions. In some embodiments, the humidity is ambient. In some embodiments, the humidity is about 10% to about 90%, e.g., about 10% to about 80%, about 10% to about 75%, about 10% to about 70%, about 10% to about 65%, about 10% to about 60%, about 20% to about 90%, about 20% to about 80%, about 20% to about 75%, about 20% to about 70%, about 20% to about 65%, about 20% to about 60%, about 30% to about 90%, about 30% to about 80%, about 30% to about 75%, about 30% to about 70%, about 30% to about 65%, or about 30% to about 60%. In some embodiments, the humidity is about 10%, about 15%, about 20%, about 25%, about 30%, about 35%, about 40%, about 45%, about 50%, about 55%, about 60%, about 65%, about 70%, or about 75%. In some embodiments, the humidity is up to 10%, up to 15%, up to 20%, up to 25%, up to 30%, up to 35%, up to 40%, up to 45%, up to 50%, up to 55%, up to 60%, up to 65%, up to 70%, or up to 75%.

In some embodiments, the composition comprising a liquid endospore suspension and at least one osmotic preservative can include about 0.01 wt % (weight percent) to 15.0 wt % osmotic preservative, e.g., about 0.01 wt % to 10.0 wt %, about 0.01 wt % to 5.0 wt %, about 0.01 wt % to 3.0 wt %, about 0.01 wt % to 1.0 wt %, about 0.1 wt % to 15.0 wt %, about 0.1 wt % to 10.0 wt %, about 0.1 wt % to 5.0 wt %, about 0.1 wt % to 3.0 wt %, about 0.1 wt % to 1.0 wt %, about 0.5 wt % to 15.0 wt %, about 0.5 wt % to 10.0 wt %, about 0.5 wt % to 5.0 wt %, about 0.5 wt % to 3.0 wt %, about 0.5 wt % to 1.0 wt %, about 1.0 wt % to 15.0 wt %, about 1.0 wt % to 10.0 wt %, about 1.0 wt % to 5.0 wt %, or about 1.0 wt % to 3.0 wt % osmotic preservative. In some embodiments, the composition can include at least about 0.01 wt %, at least about 0.05 wt %, at least about 0.1 wt %, at least about 0.5 wt %, at least about 1.0 wt %, at least about 2.0 wt %, at least about 3.0 wt %, at least about 5.0 wt %, at least about 10.0 wt %, at least about 15.0 wt %, at least about 20.0 wt %, at least about 25.0 wt %, at least about 30.0 wt %, at least about 35.0 wt %, at least about 40.0 wt %, at least about 45.0 wt %, or at least about 50.0 wt % osmotic preservative. In some embodiments, the composition comprising a liquid endospore suspension and at least one osmotic preservative can include about 0.01 wt % to 20.0 wt % osmotic preservative. In some embodiments, the composition comprising a liquid endospore suspension and at least one osmotic preservative can include about 1 wt % to 20.0 wt % osmotic preservative. In some embodiments, the composition comprising a liquid endospore suspension and at least one osmotic preservative can include about 5.0 wt % to 20.0 wt % osmotic preservative. In some embodiments, the composition comprising a liquid endospore suspension and at least one osmotic preservative can include about 5.0 wt % to 10.0 wt % osmotic preservative. In some embodiments, the composition comprising a liquid endospore suspension and at least one osmotic preservative can include about 0.01 wt % to 10.0 wt % osmotic preservative. In some embodiments, the composition comprising a liquid endospore suspension and at least one osmotic preservative can include about 0.1 wt % to 5.0 wt % osmotic preservative. In some embodiments, the composition comprising a liquid endospore suspension and at least one osmotic preservative can include about 0.1 wt % to 1.0 wt % osmotic preservative. In some embodiments, the composition comprising a liquid endospore suspension and at least one osmotic preservative can include about 0.5 wt % to 1 wt % osmotic preservative. In some embodiments, the composition comprising a liquid endospore suspension and at least one osmotic preservative can include about 0.5 wt/o to 2.0 wt % osmotic preservative. In some embodiments, the composition comprising a liquid endospore suspension and at least one osmotic preservative comprises about 1.0 wt % osmotic preservative. In some embodiments, the composition comprising a liquid endospore suspension and at least one osmotic preservative comprises about 5.0 it % osmotic preservative. In some embodiments, the composition comprising a liquid endospore suspension and at least one osmotic preservative comprises about 10.0 wt % osmotic preservative. In some embodiments, the composition comprising a liquid endospore suspension and at least one osmotic preservative comprises about 15.0 wt % osmotic preservative. In some embodiments, the composition comprising a liquid endospore suspension and at least one osmotic preservative comprises about 20.0 wt % osmotic preservative. In some embodiments, the at least one osmotic preservative comprises sodium chloride, calcium chloride, potassium chloride, magnesium chloride, or any combination thereof.

In some embodiments, the composition comprising a liquid endospore suspension and at least one osmotic preservative can include about 5 g osmotic preservative per 1 L of composition, about 6 g osmotic preservative per 1 L of composition, about 7 g osmotic preservative per 1 L of composition, about 8 g osmotic preservative per 1 L of composition, about 9 g osmotic preservative per 1 L of composition, about 10 g osmotic preservative per 1 L of composition, about 11 g osmotic preservative per 1 L of composition, about 12 g osmotic preservative per 1 L of composition, about 13 g osmotic preservative per 1 L of composition, about 14 g osmotic preservative per 1 L of composition, about 15 g osmotic preservative per 1 L of composition, about 16 g osmotic preservative per 1 L of composition, about 18 g osmotic preservative per 1 L of composition, about 20 g osmotic preservative per 1 L of composition, about 30 g osmotic preservative per 1 L of composition, about 40 g osmotic preservative per 1 L of composition, about 50 g osmotic preservative per 1 L of composition, about 60 g osmotic preservative per 1 L of composition, about 70 g osmotic preservative per 1 L of composition, about 80 g osmotic preservative per 1 L of composition, about 90 g osmotic preservative per 1 L of composition, about 100 g osmotic preservative per 1 L of composition, about 110 g osmotic preservative per 1 L of composition, about 120 g osmotic preservative per 1 L of composition, about 130 g osmotic preservative per 1 L of composition, about 140 g osmotic preservative per 1 L of composition, about 150 g osmotic preservative per 1 L of composition, or about 200 g osmotic preservative per 1 L of composition. In some embodiments, the composition comprising a liquid endospore suspension and at least one osmotic preservative can include about 50 g osmotic preservative per 1 L. In some embodiments, the composition comprising a liquid endospore suspension and at least one osmotic preservative can include about 100 g osmotic preservative per 1 L. In some embodiments, the composition comprising a liquid endospore suspension and at least one osmotic preservative can include about 150 g osmotic preservative per 1 L. In some embodiments, the at least one osmotic preservative comprises sodium chloride, calcium chloride, potassium chloride, magnesium chloride, or any combination thereof.

In some embodiments, the composition comprising the liquid endospore suspension comprises one or more additional preservative agents, i.e. in addition to the osmotic preservatives described herein, a buffering agent, suspending agent, surfactant, or a combination thereof. Exemplary additional preservative agents, buffering agents, suspending agents and surfactants are described in WO 2021/022128, the contents of which are incorporated by reference in their entirety herein.

In some embodiments, the composition comprising the liquid endospore suspension can include one or more additional preservative agents, i.e. in addition to the osmotic preservatives described herein. The composition comprising the liquid endospore suspension can include one preservative agent, two preservative agents, three preservative agents, or more, including the osmotic preservative.

In some embodiments, the composition comprising the liquid endospore suspension can include about 0.01 wt % to 15.0 wt % of the one or more additional preservative agent, e.g., about 0.01 wt % to 10.0 wt %, about 0.01 wt % to 5.0 wt %, about 0.01 wt % to 3.0 wt %, about 0.01 wt % to 1.0 wt %, about 0.1 wt % to 15.0 wt %, about 0.1 wt % to 10.0 wt %, about 0.1 wt % to 5.0 wt %, about 0.1 wt % to 3.0 wt %, about 0.1 wt % to 1.0 wt %, about 0.5 wt % to 15.0 wt %, about 0.5 wt % to 10.0 wt %, about 0.5 wt % to 5.0 wt %, about 0.5 wt % to 3.0 wt %, about 0.5 wt % to 1.0 wt %, about 1.0 wt % to 15.0 wt %, about 1.0 wt % to 10.0 wt %, about 1.0 wt % to 5.0 wt %, or about 1.0 wt % to 3.0 wt % of the one or more additional preservative agents.

The one or more additional preservative agents can be selected from a modified isothiazolin compound, an ester of p-hydroxybenzoic acid, a modified quaternary amine, a modified urea, a glycerin derivative, 2-bromo-2-nitro-1,3-propanediol, a natural oil, an organic acid having a molecular weight of no more than 200 and at least one pKa greater than 4.2, and a combination thereof.

Examples of modified isothiazolin compounds include, but are not limited to, 1,2-benzisothiazolin-3-one, methylisothiazolinone, methylchloroisothiazolinone, benzisothiazolinone, and a combination thereof.

Examples of esters of p-hydroxybenzoic acid include, but are not limited to, methylparaben, ethylparaben, propylparaben, and a combination thereof.

Examples of modified quaternary amines include, but are not limited to, benzethonium chloride or cetylpyridinium chloride, and a combination thereof.

Examples of modified urea include, but are not limited to, diazolidinyl urea, imidazolidinyl urea, and a combination thereof.

Examples of glycerin derivatives include, but are not limited to, ethylhexylglycerin.

Examples of natural oils include, but are not limited to, grapefruit seed extract, tea tree oil, thyme oil, lemongrass oil, oregano oil, rosemary oil, lavender oil, and a combination thereof.

Examples of organic acids include, but are not limited to, acetic acid, citric acid, ascorbic acid, sorbic acid, propanoic acid, butyric acid, oxalic acid, succinic acid, malic acid, tartaric acid, fumaric acid, aconitic acid, dipicolinic acid, an amino acid, and a combination thereof. Examples of amino acids include, but are not limited to, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In some embodiments, the one or more additional preservative agent comprises calcium sulfate. In some embodiments, the composition comprising liquid endospore suspension includes from about 0.01 wt % to about 5 wt % calcium sulfate. In some embodiments, composition comprising the liquid endospore suspension includes from about 0.1 wt % to about 1 wt % calcium sulfate. In some embodiments, composition comprising the liquid endospore suspension includes from about 0.2 wt % to about 1.5 wt % calcium sulfate. In some embodiments, the composition comprising the liquid endospore suspension includes about 0.25 w % calcium sulfate.

In some embodiments, the one or more additional preservative agent includes 1,2-benzisothiazolin-3-one. In some embodiments, the composition comprising the liquid endospore suspension includes about 0.01 wt % to about 2.0 wt %, about 0.01 wt % to about 1.5 wt %, about 0.01 wt % to about 1.0 wt %, or about 0.05 wt % to about 1.0 wt % 1,2-benzisothiazolin-3-one.

In some embodiments, the composition comprising the liquid endospore suspension can include about 0.01 wt % to about 0.2 wt % modified benzisothiazolin, p-hydroxybenzoic ester, modified urea, or mixtures thereof, about 0.05 wt % to about 1 wt % low molecular weight organic acid, and/or about 0.1 wt % to about 10 wt % inorganic salt. In some embodiments, the preservation solution or stable aqueous microbial composition can include about 0.01 wt % to about 0.1 wt % 1,2-benzisothiazolin-3-one, about 0.1 wt % to about 1 wt % sorbic acid, and about 0.1 wt % to 10 wt % sodium chloride.

The composition comprising the liquid endospore suspension can include about 0.01 wt % to 15.0 wt % suspending agent, e.g., about 0.01 wt % to 10.0 wt %, about 0.01 wt % to 5.0 wt %, about 0.01 wt % to 3.0 wt %, about 0.01 wt % to 1.0 wt %, about 0.1 wt % to 15.0 wt %, about 0.1 wt % to 10.0 wt %, about 0.1 wt % to 5.0 wt %, about 0.1 wt % to 3.0 wt %, about 0.1 wt % to 1.0 wt %, about 0.1 wt % to 0.5 wt %, about 0.1 wt % to 0.4 wt %, about 0.5 wt % to 15.0 wt %, about 0.5 wt % to 10.0 wt %, about 0.5 wt % to 5.0 wt %, about 0.5 wt % to 3.0 wt %, about 0.5 wt % to 1.0 wt %, about 1.0 wt % to 15.0 wt %, about 1.0 wt % to 10.0 wt %, about 1.0 wt % to 5.0 wt %, or about 1.0 wt % to 3.0 wt % suspending agent.

The composition comprising the liquid endospore suspension can include one suspending agent, two suspending agents, three suspending agents, or more. The suspending agent is used, inter alia, for suspending the microbes in the aqueous composition, thereby providing the desirable physical stability. Without wishing to be bound by theory, the suspending agent can modify the viscosity of the aqueous composition, thereby preventing settling of the microbial species to the bottom of a container.

The suspending agent can be a polymer, a surfactant, or a combination thereof. In some embodiments, the suspending agent is a polymer. Examples of polymers can include, but are not limited to, xanthan gum, guar gum, acacia gum, carboxymethylcellulose, sodium polyacrylate, polyethylene glycol, an ethylene oxide-propylene oxide (EO-PO) block copolymer, a modified starch, a modified polyacrylate, a modified methyl methacrylate, a polyethylene imine, sodium polyaspartate, poly-γ-glutamic acid, or a combination thereof. In some embodiments, the suspending agent is a blend of xanthan and acacia gums (e.g., Solagum™ AX). In some embodiments, the preservation solution or stable aqueous microbial composition can include about 0.1 wt % to 1.0 wt % polymer.

In some embodiments, the suspension agent comprises as rheological modifier. Rheological modifiers are substances that alter the rheological properties of a material, i.e. that are added to a formulation to alter viscosity. Suitable rheological modifiers include, but are not limited to, commercially available rheological modifiers comprising urea and modified versions of urea, such as RHEOBYK®-410. Other rheological modifiers will be known to persons of ordinary skill in the art.

Examples of surfactants include, but are not limited to, a primary alkyl alcohol ethoxylate, a secondary alkyl alcohol ethoxylate, a primary alkyl alcohol propoxylate, a secondary alkyl alcohol propoxylate, and a combination thereof. In some embodiments, the surfactant has a cloud point from about 30° C. to about 80° C., and hydrophilic-lipophilic balance from about 5 to about 15. In some embodiments, the surfactant is a C-13 branched primary alcohol with average ethoxylation of 5 to 10, e.g., 5, 6, 7, 8, 9, or 10.

In some embodiments, the surfactant includes an ethoxylated primary branched Cl3 alcohol with full saturation, such as Synperonic™ 13/7 or Synperonic™ 13/6. In some embodiments, the surfactant includes alcohol ethoxylate, such as Ecosurf™ EH-6. In some embodiments, the surfactant includes a mixture of 58.0-62.0% D-glucopyranose, oligomeric, decyl octyl glycoside and 38.0-42.0% water, and contains less than 2% decanol and less than 1.0% octanol, such as Triton™ CG110. In some embodiments, the surfactant includes secondary polyether polyol, such as Tergitol™ L-62. In some embodiments, the surfactant includes secondary alcohol ethoxylate, such as Tergitol™ 15-S-12. In some embodiments, the surfactant includes a non-ionic alkyl EO/PO copolymer, such as Tergitol™ XDLW.

In some embodiments, the liquid endospore suspension can include about 0.1 wt % to 10 wt % surfactant, e.g., about 0.1 wt % to 5 wt %, 0.1 wt % to 2 wt %, 0.1 wt % to 1 wt %, 0.5 wt % to 1 wt %, or 0.5 wt % to 2 wt % surfactant.

In some embodiments, the suspending agent can include xanthan gum, acacia gum, and alcohol ethoxylate.

The composition comprising the liquid endospore suspension can include about 0.01 wt % to 15.0 wt % buffering agent, e.g., about 0.01 wt % to 10.0 wt %, about 0.01 wt % to 5.0 wt %, about 0.01 wt % to 3.0 wt %, about 0.01 wt % to 1.0 wt %, about 0.1 wt % to 15.0 wt %, about 0.1 wt % to 10.0 wt %, about 0.1 wt % to 5.0 wt %, about 0.1 wt % to 3.0 wt %, about 0.1 wt % to 1.0 wt %, about 0.5 wt % to 15.0 wt %, about 0.5 wt % to 10.0 wt %, about 0.5 wt % to 5.0 wt %, about 0.5 wt % to 3.0 wt %, about 0.5 wt % to 1.0 wt %, about 1.0 wt % to 15.0 wt %, about 1.0 wt % to 10.0 wt %, about 1.0 wt % to 5.0 wt %, or about 1.0 wt % to 3.0 wt % buffering agent.

The buffering agent can be in an amount sufficient to keep the pH of the composition comprising the liquid endospore suspension at greater than 4.2, e.g., greater than 4.5, greater than 5.0, greater than 5.5, greater than 6.0, greater than 6.5, or greater than 7.0. In some embodiments, the buffering agent can be in an amount sufficient to keep the pH of the composition comprising the liquid endospore suspension in the range of about 4.3 to about 12.0, e.g., about 4.3 to about 11.0, about 4.3 to about 10.0, about 4.3 to about 9.5, about 4.3 to about 9.0, about 4.3 to about 8.5, about 5.0 to about 12.0, about 5.0 to about 11.0, about 5.0 to about 10.0, about 5.0 to about 9.5, about 5.0 to about 9.0, about 5.0 to about 8.5, about 5.5 to about 12.0, about 5.5 to about 11.0, about 5.5 to about 10.0, about 5.5 to about 9.5, about 5.5 to about 9.0, or about 5.5 to about 8.5.

The pH of the composition comprising the liquid endospore suspension can be selected to maximize shelf life of the spores and/or colonies stored in the aqueous composition for extended periods. pH's less than about 4 may have a detrimental effect on stability of certain bacterial spores at elevated temperatures. For example, Bacillus spores can demineralize at low pH, losing their resistance to high temperature storage. Therefore, a pH greater than about 4.2, about 4.5, about 5.0, about 5.5, or about 6.0 is desirable for improving the biological stability of Bacillus spores. In some embodiments, the pH of the composition comprising the liquid endospore suspension is greater than about 6.0.

In some aspects, the present disclosure relates to methods of using the compositions of the present disclosure for inhibition of the proliferation of at least one environmental spoilage organism. Compositions of the present disclosure can be antagonistic to spoilage organisms while simultaneously supportive of endospore dormancy and stability.

Another aspect of the present disclosure relates to a method of inhibiting the proliferation of at least one environmental spoilage organism in a liquid endospore suspension, wherein the method comprises use of at least one osmotic preservative.

In some embodiments, the method comprises inhibition of the proliferation of at least one environmental spoilage organism in a composition comprising a liquid endospore suspension by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, the inhibition of the proliferation of at least one environmental spoilage organism in a liquid endospore suspension is about 10% to about 95%, e.g., about 10% to about 85%, about 10% to about 75%, about 10% to about 70%, about 10% to about 65%, about 10% to about 60%, about 20% to about 95%, about 20% to about 85%, about 20% to about 75%, about 20% to about 70%, about 20% to about 65%, about 20% to about 60%, about 30% to about 95%, about 30% to about 85%, about 30% to about 75%, about 30% to about 70%, about 30% to about 65%, or about 30% to about 60%. In some embodiments, the inhibition of the proliferation of at least one environmental spoilage organism in a liquid endospore suspension is about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, inhibition of the proliferation of at least one environmental spoilage organism is measured relative to the proliferation of the at least one environmental spoilage organism in an otherwise equivalent composition comprising the liquid endospore suspension lacking the at least one osmotic preservative.

In some embodiments, the at least one environmental spoilage organism includes, but is not limited to a bacterial, a yeast, or a fungus contaminant. In some embodiments, the at least one environmental spoilage organism comprises a bacterial contaminant, a yeast contaminant, a fungus contaminant or any combination thereof. In some embodiments, the bacterial contaminant comprises Pseudomonas species, Citrobacter species, Klebsiella species, Shigella species, Enterobacter species, Serratie species, or any combination thereof.

In some embodiments, the method comprises inhibition of the proliferation of at least one environmental spoilage organism in a composition comprising a liquid endospore suspension comprising endospore forming Bacillota. In some embodiments, the endospore forming Bacillota is a Bacillus species, Brevibacillus species, Paenibacillus species, Lysinibacillus species, or any combination thereof.

In some embodiments, the method comprises inhibition of the proliferation of at least one environmental spoilage organism in a composition comprising a liquid endospore suspension comprising a Bacillus species. In some embodiments, the Bacillus species comprises B. amyloliquefaciens (BA), B. subtilis (BS), B. pumilus (BP), B. licheniformis (BL). B. thuringiensis. B. velezensis, B. endophyticus, Priestia megaterium (basonym Bacillus megaterium), another Bacillus species, or any combination thereof.

In some embodiments, the aforementioned methods comprise the use of at least one osmotic preservative. In some embodiments, the at least one osmotic preservative is an osmolarity modifier. In some embodiments, the at least one osmotic preservative is a combination of osmolarity modifiers. In some embodiments, the osmolarity modifier increases osmolarity within the liquid endospore suspension. In some embodiments, the osmolarity modifier decreases osmolarity within the liquid endospore suspension. In some embodiments, the osmolarity modifier reduces water energy and/or induces preservation of the endospores within the liquid endospore suspension.

In some embodiments, the aforementioned methods comprise the use of a preservative system. In some embodiments, the preservative system can include at least one osmotic preservative, at least two osmotic preservatives, at least three osmotic preservatives, or four or more osmotic preservatives. In some embodiments, the preservative system can include at least one osmolarity modifier, at least two osmolarity modifiers, at least three osmolarity modifiers, or four or more osmolarity modifiers. Alternatively, in some embodiments, the preservative system can be a chemical and/or physical preservative system.

In some embodiments, the at least one osmotic preservative is a salt or a combination of salts. In some embodiments, the at least one osmotic preservative is one or more chloride salts. In some embodiments, the at least one osmotic preservative is sodium chloride, calcium chloride, potassium chloride, magnesium chloride, or any combination thereof. In some embodiments, the osmotic preservative increases osmolarity within the liquid endospore suspension. In some embodiments, the osmotic preservative decreases osmolarity within the liquid endospore suspension. In some embodiments, the osmotic preservative reduces water energy and/or induces preservation of the endospores within the liquid endospore suspension.

In some embodiments, the preservative system can include at least one osmotic preservative, at least two osmotic preservatives, at least three osmotic preservatives, or four or more osmotic preservatives. In some embodiments, the preservative system can include at least one salt, at least two salts, at least three salts, or four or more salts. Alternatively, in some embodiments, the preservative system can be a chemical and/or physical preservative system.

When used in agronomic applications, the compositions of the present disclosure can provide a number of desirable characteristics related to decreased spoilage and endospore stability, including, but not limited to, the antagonistic effect to spoilage organisms and the supportive effect of endospore dormancy and stability.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” Any ranges cited herein are inclusive.

The terms “substantially”, “approximately,” and “about” used throughout this Specification and the claims generally mean plus or minus 10% of the value stated, e.g., about 100 would include 90 to 110.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B): in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” refers to weight percent.

As used herein, “preservative system” refers to one or more osmotic preservatives or at least one osmotic preservative. Further, a preservative system prohibits the proliferation of spoilage organisms in a liquid endospore suspension without compromising the viability of the product's formulated endospores. Proliferation of spoilage organisms can be measured using known methods in the art. For example, in the pharmaceutical and personal care industries, USP 41/51 requires a reduction in challenge microbe populations of two orders of magnitude in order for a preservative to be deemed efficacious.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, the term “biologically stable” refers to the microbial titer of an aqueous microbial composition changing no more than 25% (e.g., no more than 20%, no more than 15%, or no more than 10%) upon storage at room temperature for a period of time, e.g., at least 6 months. In some embodiments, the term “biologically stable” refers to the microbial titer remaining at or above the relevant label claim.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Examples

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Preparation of Bacillus Endospores

The microbes of the present disclosure can be grown using standard submerged liquid fermentation processes known in the art.

Individual starter cultures of Bacillus subtilis, Bacillus licheniformis. Bacillus amyloliquefaciens, and Bacillus pumilus were grown according to the following general protocol and adapted as required for each organism: 2 grams Nutrient Broth, 2 grams AmberFerm (yeast extract) and 4 grams maltodextrin are added to a 250 mL Erlenmeyer flask. 100 mLs distilled, deionized water were added and the flask was stirred until all dry ingredients dissolve. The flask was covered and placed for 30 minutes in an autoclave operating at 121° C. and 15 psi. After cooling, the flask was inoculated with 1 mL of one of the pure microbial strains. The flask was sealed and placed on an orbital shaker at 30° C. Cultures were allowed to grow for 3-5 days. This procedure was repeated (or performed in parallel) for each organism.

Larger cultures were prepared by adding 18 grams Nutrient Broth, 18 grams AmberFerm, and 36 grams maltodextrin to 1-liter flasks with 900 mLs distilled, deionized water. The flasks were sealed and sterilized as above. After cooling, 100 mLs of the microbial media from the 250 mL Erlenmeyer flasks was added. The 1-liter flasks were sealed, placed on an orbital shaker, and allowed to grow for another 3-5 days at 30° C.

In the final grow-out phase before introduction to the fermenter, cultures from the 1-liter flasks were transferred under sterile conditions to sterilized 6-liter vessels, and fermentation continued at 30° C. with aerating until stationary phase was achieved. The contents of each 6-liter culture flask were transferred to individual fermenters which are also charged with a sterilized growth media made from 1 part yeast extract and 2 parts dextrose. The individual fermenters were run under aerobic conditions operating at pH 7.0 and the temperature optimum for each species.

Once cell density reached 10¹¹ CFU/mL, the fermenters were heat shocked at >80° C. to induce spore formation. Spores were recovered from the liquid fermentation media via filtration then resuspended in an aqueous medium at a titer of about 10¹¹ CFU/mL. Each stock aqueous suspension of pure Bacillus spores was stored cold (˜4° C.).

A Bacillus concentrate with a final titer greater than about 40×10¹⁰ CFU/mL was prepared by mixing the individual stock aqueous suspensions of Bacillus subtilis, Bacillus licheniformis, Bacillus amyloliquefaciens, and Bacillus pumilus into an aqueous medium stabilized with 0.25 wt % calcium sulfate.

### Example 2. Stability of Liquid Bacillus Endospore Suspension

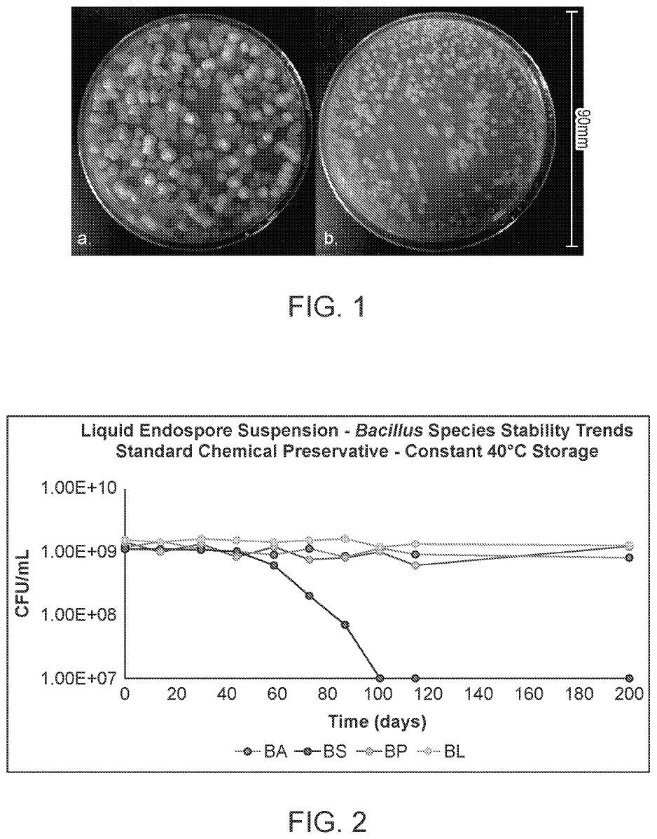

In one experiment, a variety of bacteria were isolated from samples of contaminated product, and culturable microbial communities differed in species compositions (J. P. Gorsuch, Z. Jones, Heliyon 6 (2020) e03419). Plates of MacConkey's agar were prepared from serial dilutions of two separate contaminated product samples from southern Vietnam showing qualitative differences in colony morphology among their dominant cultural species. As shown in FIG. 1, members of the Bacillus are subject to instability mechanisms, such as spoilage by environmental microorganisms. Without wishing to be bound by theory, the results from the experiment described above, i.e. the spoilage organisms recovered from samples of a commercially available endospore suspension protected with a standard chemical preservative and exposed to standard unit operations at a customer's facility, supports the need to include an advanced antimicrobial inhibitor system in future endospore suspension products to eliminate or decrease the amount of spoilage organisms.

In a second experiment, the stability of a blend of four industrially-relevant Bacillus species (Bacillus amyloliquefaciens (BA), B. subtilis (BS), B. pumilus (BP), and B. licheniformis (BL)) was determined under stressed conditions in the presence of a standard chemical preservative. Standard chemical preservatives include, but are not limited to. Proxel™ and Bioban™. Sub-populations of individual species within the blend were assessed using a customized spread-plate assay with morphological scoring (Gorsuch, J., Journal of Microbiological Methods 164, 105682, 2019) which allowed separate reporting of each population from a single tested blend. As shown in FIG. 2, one Bacillus species, B. subtilis (BS), is subject to instability mechanisms, such as loss of endospore viability over time under stressed storage conditions. Without wishing to be bound by theory, the results from the experiment described above demonstrates that while BA, BP, and BL retain viability, BS loses viability rapidly and falls below the lower limit of detection for the assay after 100 days of storage.

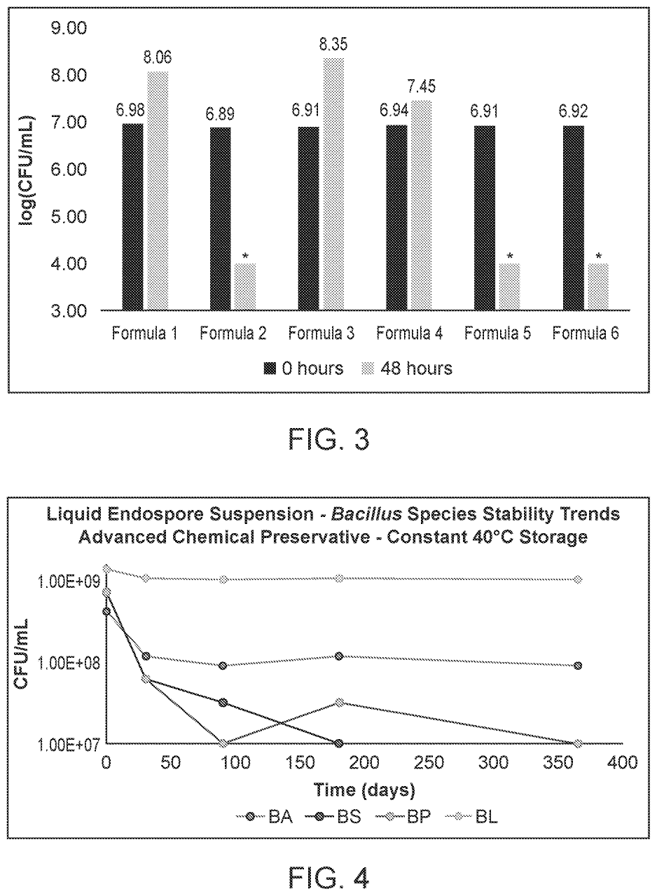

In a third experiment, the stability of a blend of four industrially-relevant Bacillus species (Bacillus amyloliquefaciens (BA), B. subtilis (BS), B. pumilus (BP), and B. licheniformis (BL)) with advanced chemical preservation packages (formulas 1-6 in FIG. 3) was determined. As shown in FIG. 3, the stability was assessed by determining the ability of the blend to mitigate spoilage by environmental microorganisms using a customized preservative efficacy test (J. P. Gorsuch, Z. Jones, Heliyon 6 (2020) e03419). As shown in FIG. 4, sub-populations of individual species within the blend were assessed using a customized spread-plate assay with morphological scoring (Gorsuch, J., Journal of Microbiological Methods 164, 105682, 2019) allowing separate reporting of each population from a single tested blend. While advanced chemical preservative packages adequately protect the product from spoilage by environmental contaminants (FIG. 3), they also have a negative effect over time on endospore stability for three out of the four Bacillus species (BA, BS, and BP) (FIG. 4). Accordingly, over time the product failed meet its label claims. Without wishing to be bound by theory, the results from the experiment described above demonstrate that a preservative system is needed which can mitigate the threat of spoilage posed by hardy environmental contaminants while also improving stability outcomes for labile Bacillus endospores.

### Example 3. Stability of Liquid Bacillus Endospore Suspension with an Osmotic Preservative System

Osmotic preservative strategies based on salts such as sodium chloride (NaCl) and potassium chloride (KCl) are used in the food production industry to protect substrates of interest from microbial spoilage (Pizarro, J., Revista Argentina de Microbiologia, Volume 50, Issue 1, pp. 56-61). The mechanism of action for these salt-based systems is understood to be disruption of cellular structure and function (Gandhi A, J Dairy Sci. 2014, 97(10):5939-51). However, Bacillus endospores are naturally dehydrated (Setlow, P., 2014, Bacteriol. 196, 1297-1305) and as such would be impervious to this type of cellular disruption. Additionally, in the spores of some Bacillus species, sodium chloride (NaCl) is known to inhibit nutrient and non-nutrient-germination responses (Nagler K, Appl Environ Microbiol. 2014; 80(4):1314-1321). There is no osmotic preservation package available which generates both effects—prevention of product spoilage and a stabilization of endospore viability for otherwise labile species.

In one experiment, prototype endospore suspensions were screened to identify osmolyte additives which produce the set of parameters discussed above (Table 1). Each prototype was a mixed-species assemblage consisting of equivalent concentrations of BA, BS, BP, and BL endospores. Each species represented a testable sub-population within the overall assemblage, and each population was assessed separately over time using a customized spread-plate assay with morphological scoring allowing separate reporting of each population from a single tested blend.

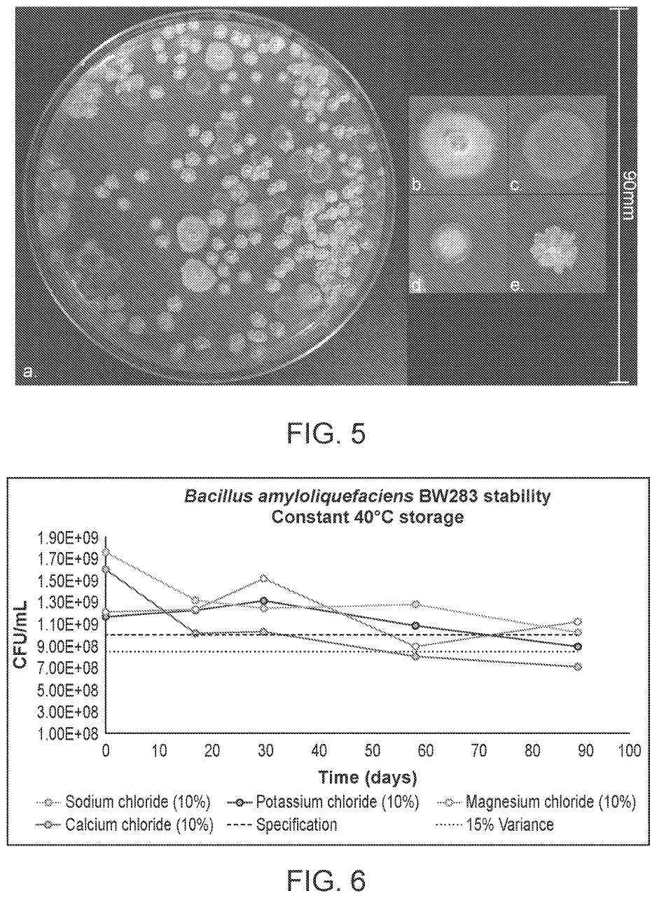

A range of industrially relevant solutes (sodium chloride, magnesium chloride, potassium chloride, and calcium chloride, respectively) were screened for the ability to prevent product spoilage and mitigate endospore instability. Samples were stored under stressed environmental storage conditions (constant 40° C.). Preservative efficacy was assessed at 0, 30, 59, and 90 days of storage (sampling ongoing) using a published PET assay (J. P. Gorsuch, Z. Jones, Heliyon 6 (2020) e03419). Results were compared with a positive control composed of the advanced chemical preservation package described above. Populations of individual endospores were assessed at 0, 14, 30, 60, 90, 120, and 150 days of storage (sampling ongoing) using a published plate counting assay which includes morphological scoring of individual species within the microbial assemblage (FIG. 5) allowing separate enumeration of each population within the assemblage over time.

TABLE 1
Composition of prototype endospore suspensions.

| | NaCl | KCl | MgCl₂ | CaCl₂ |
|---|---|---|---|---|
| | prototype | prototype | prototype | prototype |
| Bacillus | 25 | 25 | 25 | 25 |
| amyloliquefaciens | | | | |
| concentrate (mL) | | | | |
| Bacillus subtilis | 25 | 25 | 25 | 25 |
| concentrate (mL) | | | | |
| Bacillus pumilus | 25 | 25 | 25 | 25 |
| concentrate (mL) | | | | |
| Bacillus licheniformis | 25 | 25 | 25 | 25 |
| concentrate (mL) | | | | |
| NaCl (grams) | 100 | — | — | — |
| KCl (grams) | — | 100 | — | — |
| MgCl₂ (grams) | — | — | 100 | — |
| CaCl₂ (grams) | — | — | — | 100 |
| Water (mL) | 800 | 800 | 800 | 800 |

The ability of various osmotic preservative packages to inhibit the proliferation of environmental spoilage organisms were examined using customized PET assays (Table 2). The displayed values represent fold changes in the population of bacterial and yeast contaminants, respectively, relative to their initial inoculated levels at the beginning of the assay. Without wishing to be bound by theory, any negative result is considered passing, a greater negative value indicates a more aggressive microbicidal effect. Without wishing to further be bound by theory, the results from the experiment described above demonstrates that NaCl and CaCl₂ tend to have less aggressive effects than KCl and MgCl₂, suggesting a less robust antimicrobial effect.

TABLE 2
Customized PET assay examining inhibition of the proliferation of environmental spoilage organisms.

| | 0 | | 30 | | 59 | | 90 | | Average | |
|---|---|---|---|---|---|---|---|---|---|---|
| | Bacterial | Yeast | Bacterial | Yeast | Bacterial | Yeast | Bacterial | Yeast | Bacterial | Yeast |
| Control | −5.62 | −2.1 | −5.26 | −1.5 | −5.54 | −2.08 | — | — | −5.47 | −1.89 |
| (current FC) | | | | | | | | | | |
| KCl | −5.71 | −2.07 | −2.26 | −0.33 | −5.5 | −2.08 | −1.05 | −2.05 | −3.63 | −1.63 |
| NaCl | −0.76 | −0.69 | −1.08 | −1.53 | −0.87 | 0 | −1.21 | −1.3 | −0.98 | −0.88 |
| MgCl2 | −5.41 | −2.12 | −4.97 | −1.51 | −4.83 | −0.42 | −5.44 | −2.07 | −5.16 | −1.53 |
| CaCl2 | −2.38 | −0.09 | −2.25 | −1.79 | −2.58 | −0.03 | −1.51 | −2.09 | −2.18 | −1 |

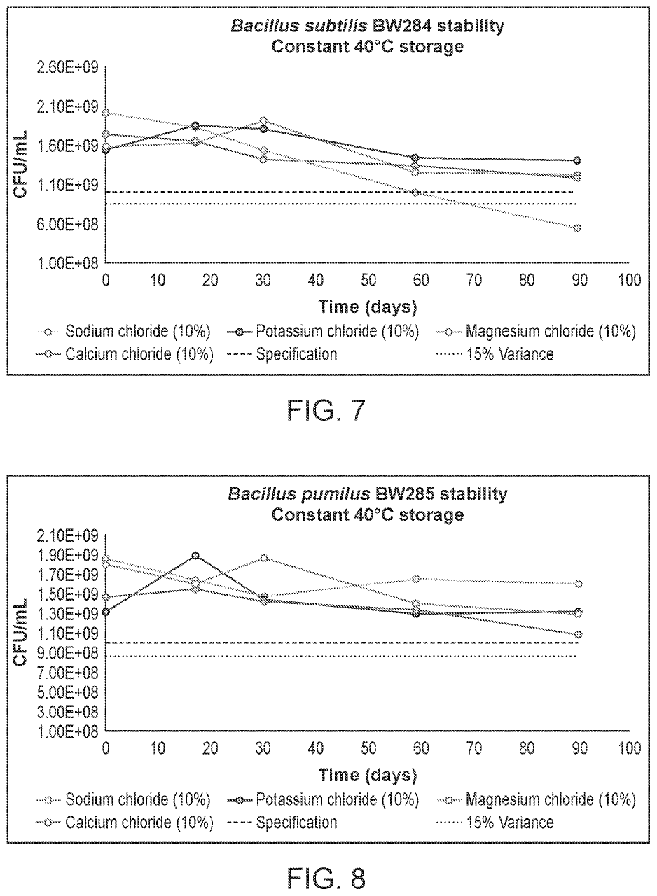

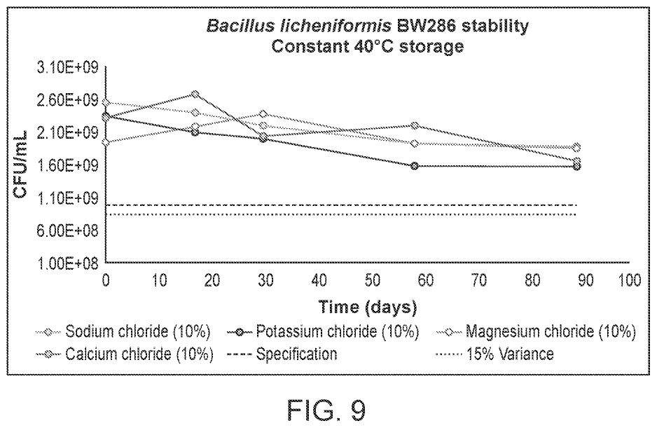

The results of Bacillus endospore stability studies, collected from the mixtures described in Table 1, are presented in separate graphs (FIGS. 6-9). Each individual species represented a testable sub-population within the mixture via morphological scoring. The stability of BA populations (FIG. 6) within the tested blends (Table 1) compared favorably with historical data (FIG. 1) in all treatments except those preserved with CaCl₂). The stability of BS populations (FIG. 7) within the tested blends (Table 1)—historically unstable under these storage conditions—presented the same instability profile (FIG. 1) only in treatments preserved with NaCl, while treatments preserved with KCl, MgCl₂ and CaCl₂), respectively, showed a positive stability profile not previously observed for this species. The stability of BP and BL populations (FIG. 8 and FIG. 9, respectively) within the tested blends (Table 1) compared favorably with historical data (FIG. 1) in all treatments.

As discussed above, the optimal preservative system for a liquid endospore suspension produces a physical and chemical environment within the suspension that is antagonistic to spoilage organisms while simultaneously supportive of endospore dormancy and stability. The PET assay data (Table 2) shows that MgCl₂ and KCl produce the strongest antimicrobial effect against field-relevant spoilage organisms in the tested Bacillus assemblage. The endospore stability data (FIG. 6) shows that endospore stability—as assessed by culturability in vitro—is preserved for all Bacillus species only in formulations preserved with MgCl₂ and KCl. Without wishing to be bound by theory, the results demonstrate that these materials produce a physical and chemical environment which meets the criteria laid out above (i.e. antagonistic to spoilage organisms while simultaneously supportive of endospore dormancy and stability).

Without wishing to be further bound by theory, the results demonstrate that it is possible that the mechanism behind spoilage prevention may be different than the mechanism behind endospore stabilization, leading to a parallel set of parameters which must be satisfied to produce an optimal endospore suspension. For example, the antimicrobial efficacy effect may be mediated by the solution's water activity or osmolarity, or by interactions of specific cations with the cells of spoilage organisms. While the endospore stabilization effect may be mediated by the solution's water activity or osmolarity, by interactions of specific cations with endospore structures, or through chemical fixation of non-nutrient germinants in the suspension's supernatant.

Without wishing to be bound by theory, the results demonstrate that a composition comprising endospore forming Bacillota and an osmotic preservative system produces a physical and chemical environment within a liquid Bacillus formulation which is simultaneously conducive to endospore stability under stressed conditions and antagonistic to environmental spoilage organisms.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A composition comprising a liquid endospore suspension and at least one osmotic preservative.

2. The composition of claim 1, wherein the liquid endospore suspension is an aqueous suspension.

3. The composition of claim 1, wherein the liquid endospore suspension comprises at least one endospore forming Bacillota.

4. The composition of claim 1, wherein the at least one endospore forming Bacillota is selected from the group consisting gf Bacillus, Brevibacillus, Paenibacillus, and Lysinibacillus genera.

5. The composition of claim 1, wherein the Bacillus species is selected from the group consisting of B. amyloliquefaciens (BA), B. subtilis (BS), B. pumilus (BP), B. licheniformis (BL), B. thuringiensis, B. velezensis, B. endophyticus, and Priestia megaterium (basonym Bacillus megaterium).

6. The composition of claim 1, wherein the at least one osmotic preservative is an osmolarity modifier.

7. The composition of claim 6, wherein the osmolarity modifier reduces water energy and/or induces preservation of the endospores.

8. The composition of claim 1, wherein the at least one osmotic preservative is a chloride salt.

9. The composition of claim 8, wherein the at least one osmotic preservative is selected from sodium chloride, calcium chloride, potassium chloride, magnesium chloride and any other chloride salt.

10. The composition of claim 1, wherein the composition comprises at least about 10.0 wt % of the at least one osmotic preservative.

11. The composition of claim 1, wherein the liquid endospore suspension is stable at 40° C. for at least 180 days.

12. A method of inhibiting the proliferation of at least one environmental spoilage organism in a liquid endospore suspension, wherein the method comprises use of at least one osmotic preservative.

13. The method of claim 12, wherein the at least one environmental spoilage organism is selected from a bacterial, a yeast, or a fungus contaminant.

14. The method of claim 13, wherein the bacterial contaminant is selected from Pseudomonas, Citrobacter, Klebsiella, Shigella, Enterobacter, and Serratie species.

15. The method of claim 12, wherein the at least one endospore forming Bacillota is selected from Bacillus, Brevibacillus, Paenibacillus, and Lysinibacillus species.

16. The method of claim 12, wherein the Bacillus species is selected from B. amyloliquefaciens (BA), B. subtilis (BS), B. pumilus (BP), B. licheniformis (BL), B. thuringiensis, B. velezensis, B. endophyticus, and Priestia megaterium (basonym Bacillus megaterium).

17. The method of claim 12, wherein the at least one osmotic preservative is an osmolarity modifier.

18. The method of claim 17, wherein the osmolarity modifier reduces water energy and/or induces preservation of the endospores.

19. The method of claim 12, wherein the at least one osmotic preservative is selected from the group consisting of sodium chloride, calcium chloride, potassium chloride, magnesium chloride and any other chloride salt.

20. The method of claim 12, wherein the liquid endospore suspension is stable at 40° C. for at least 180 days.
